(19) United States
(12) Patent Application Publication
(10) Pub. No.: US 2007/0025510 A1
(43) Pub. Date: Feb. 1, 2007

(54) Method and apparatus for x-ray imaging of a patient during a shockwave treatment

(76) Inventors: Gerhard Buchholtz, Erlangen (DE); Jens Fehre, Hausen (DE); Bernd Granz, Oberasbach (DE); Martin Hoheisel, Erlangen (DE); Werner Kruft, Erlangen (DE); Markus Lanski, Furth (DE); Matthias Mahler, Erlangen (DE); Christian Meinert, Marloffstein (DE); Thomas Mertelmeier, Erlangen (DE); Ralf Nanke, Neunkirchen (DE); Manfred Rattner, Grossenseebach (DE)

Correspondence Address: SCHIFF HARDIN, LLP;PATENT DEPARTMENT, 6600 SEARS TOWER, CHICAGO, IL, 60606-6473, US

(21) Appl. No.: 11/479,905

(22) Filed: Jun. 30, 2006

(30) Foreign Application Priority Data
Jul. 4, 2005 (DE) 10 2005 031 123.7

(51) Int. Cl. A61N 5/10

(52) U.S. Cl. 378/65

(57) ABSTRACT

In a method for x-ray imaging given a patient containing a subject to be represented during a shockwave treatment, an image data set containing the subject and a marker is generated at a first point in time; an x-ray image showing essentially only the subject (14) and the marker is acquired at a second point in time, the x-ray image is correctly spatially associated with the image data set using the marker, the x-ray image is displayed together with information extracted from the image data set during the shockwave treatment. An apparatus for x-ray imaging a patient containing a subject to be represented during a shockwave treatment has a memory for an image data set generated at a first point in time and containing the subject and a marker, an x-ray system for acquisition of an x-ray image showing essentially only the subject and the marker at a second point in time; an evaluation unit for spatially-accurate association of the x-ray image with the image data set using the marker and for extraction of information from the image data set, and a display unit for display of the x-ray image together with the information during the shockwave treatment.

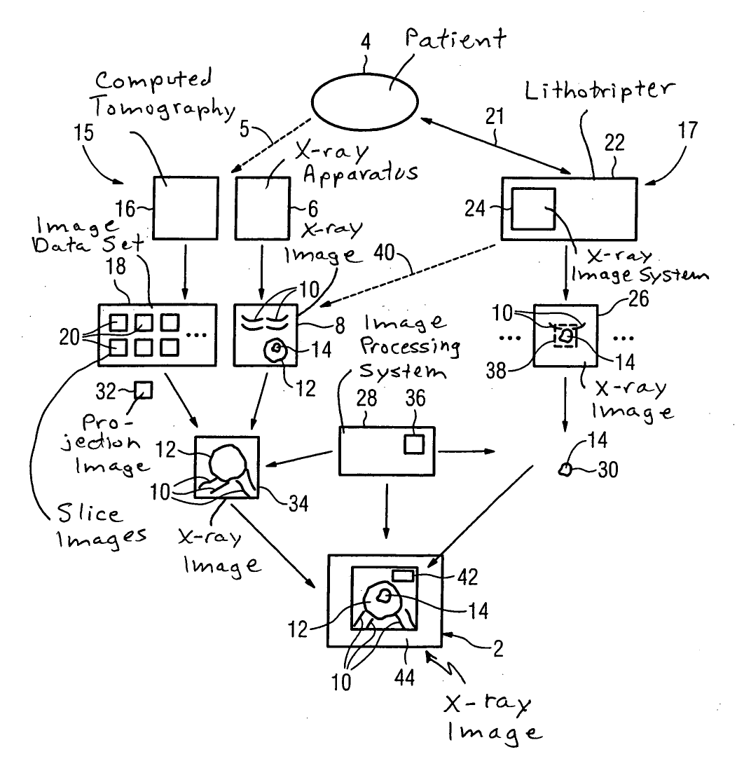

### BACKGROUND OF THE INVENTION

1. Field of the Invention

The present invention concerns a method and an apparatus for x-ray imaging OF a patient containing a subject to be represented during a shockwave treatment.

2. Description of the Prior Art

Imaging methods are used today in many fields of medicine. One of the most widespread methods is x-ray imaging, in which a living person or animal as a patient is irradiated with ionizing x-ray radiation. The goal is the representation or imaging of a subject of interest located in the patient. This can, for example, an internal organ, a foreign body or the like.

In addition to the many known advantages of x-ray imaging there are certain disadvantages associated therewith. Due to the danger of radiation injury, exposure to x-ray radiation is not risk-free for the patient or for the personnel conducting the x-ray exam. The x-ray dose radiated on a living patient is therefore legally limited to maximum values for health reasons, for example.

One of the most important goals in the use of x-rays is therefore always reducing the dose to a minimum possible quantity. This is particularly the case in x-ray-intensive medical measures in which a number of x-ray exposures of an individual patient is necessary within a short time span. Such a situation occurs, for example, in shockwave treatment in the form of shockwave therapy and shockwave lithotripsy. In the case of lithotripsy, both the position of a calculus to be disintegrated and its degree of destruction must be monitored in the patient by a number of x-ray acquisitions during the course of treatment.

In spite of the fact that the individual dose in one modern x-ray acquisition has already been drastically reduced relative to earlier x-ray techniques, in the case of a shockwave treatment, the treated patient and, to a certain degree, also the medical personnel are exposed to a high radiation exposure.

The subject of interest located in the patient that is to be represented by radiography normally occupies only a small part of the actual x-ray image. Nevertheless, the subject and its surrounding region of the patient are always imaged again with a full dose during the treatment. The remaining image information in addition to the subject is necessary because, for example, the position of the subject relative to internal organs or bone structures is visible in such an exposure. This is important in lithotripsy, for example, in order to be able to select the radiation direction or the radiation location of the ultrasound beam that is used to disintegrate the calculus.

### SUMMARY OF THE INVENTION

An object of the present invention to provide an improved method and apparatus for x-ray imaging during a shockwave treatment.

The invention is based on the recognition the realization that in many cases an image data set, for example in the form of x-ray exposures of the patient, has already been produced before implementation of the treatment, this image data set containing supplementary information (such as, for example, about the subjects such as organs or bones surrounding the subject of interest) in addition to the subject of interest itself.

The supplementary information are for the most part also still valid during the treatment because often only the subject of interest changes in the course of the treatment, for example in terms of its shape and position, but not its surroundings such as the aforementioned internal organs or bone structures. Much information acquired beforehand thus has validity for the entire treatment and does not need to be repeatedly acquired.

The subject to be represented is normally shown in advance of the shockwave treatment in sufficient image quality so that, for example, diagnosis, localization and characterization of the subject could be implemented sufficiently well before the treatment. In contrast to this, for acquisition of the subject during the treatment often a substantially reduced image quality would result in no losses for the performance of the treatment since it need only be checked whether the stone is still situated in the focus of the shockwave, but it is not longer necessary to image the stone in precise detail.

The above object with regard to the method is achieved in accordance with the invention by a method for x-ray imaging of a patient containing during a shockwave treatment a subject to be represented wherein an image data set including the subject and a marker is generated at a first point in time, an x-ray image showing essentially only the subject and the marker is acquired at a second point in time; the x-ray image is spatially correctly associated with the image data set using the marker, and the x-ray image is displayed together with information extracted from the image data set during the shockwave treatment.

Everything that is subject to visible changes in the x-ray image between the points in time of the acquisitions of image data set and x-ray image is to be understood as a subject to be represented in the sense of the present invention. In the example of lithotripsy, this would be the calculus to be destroyed, which shrinks, fragments or shifts in the course of the lithotripsy. A tissue region to be treated that changes due to displacement or size change is also possible.

The image data set includes image information that exceeds the subject to be represented, for example the aforementioned images of tissue surrounding the subject to be shown, bone structures and most of all images of natural or artificial markers for position detection or location. As already explained above, most image parts of the image data set do not change or their variations are irrelevant for the treatment. It is therefore sufficient to acquire this image data set of the patient at a first point in time in order to record the cited information once.

To acquire information about the subject of interest to be represented, as already explained it is therefore not necessary to map the information just mentioned in a detailed x-ray image at a later point in time. According to the invention, the second x-ray image is therefore acquired such that it essentially shows only the subject and the marker. The second x-ray image therefore can be acquired at a significant dose reduction as explained further below.

The mapping of the subject in the x-ray image is sufficient in order to obtain the necessary current information about the subject, and the mapping of the marker serves to correctly spatially associate the x-ray image with the image data set.

Further image information (for example surrounding the subject) that is still valid and unchanged at the second point in time is extracted from the image data set and thus is in fact mapped at an earlier first point in time, but is still entirely valid at the second point in time as explained above.

The marker serves for what is known as registration of image data set and x-ray image. The registration leads to these being correctly associated with one another in terms of position.

The marker can be a natural marker of the patient (for example a bone structure, organ boundary) or be an artificially adhered or implanted marker on the patient. The registration is effected in a known manner via a 3D image calculation system.

By the repeated acquisition of second x-ray images at respectively different second points in time that are all displayed together with the information extracted from the image data set (in other words with the information supplementing the x-ray image), a series of x-ray images is created with a significant dose reduction for the patient with the same information content as if all second x-ray images were acquired with a full dose.

Both the patient and the treating personnel are protected by the significant dose reduction in the acquisition of the second x-ray image.

In the field of shockwave lithotripsy, the first point in time can be before the beginning of the treatment. The various second points in time then lie in the time span during the lithotripsy.

The spatial position of the subject in the patient can be displayed as information.

This information is initially not to be learned from the x-ray image, but it is reconstructed by the association with the image data set using the markers. For example, it can be provided to the doctor via the display, which is how he or she receives the same information as in the case of a full-fledged x-ray image according to the prior art.

Image information of the surroundings of the subject that are not shown in the x-ray image can also be displayed as information.

Not only the extracted information but also the “missing” image information is thus provided, for example to the doctor. He or she can evaluate this information more easily and in a more familiar manner than, for example, the specification of spatial coordinates as information.

For the x-ray image, various alternatives are available for dose reduction.

For example, the x-ray image can be generated with a dose so low that the subject and the marker are still immediately recognizable. In this context, recognizable means that a viewer or an image evaluation unit can immediately extract the information that is still of interest from the x-ray image. In the case of a kidney stone, this is its contour shape in order to determine its degree of destruction and its position.

Most notably, a clearly recognizable subject or a subject delimited from its surroundings, such as a calculus to be destroyed in lithotripsy, can be clearly detected with sufficient clarity even in x-ray exposures generated with a very low dose. The information of, for example, the surroundings of the subject to be represented are then, however, no longer recognizable. The surroundings are then extracted from the image data set according to the invention and shown with the x-ray image.

Alternatively, the x-ray image can be acquired with so small an image field that the subject and the marker are still directly recognizable. This can be implemented with suitable diaphragms. The reduction of the image field and therewith the surface irradiated on the patient reduces the x-ray dose which is emitted on the patient. Although the total dose for the patient is likewise reduced, the typical dose nevertheless is provided on the limited surface for imaging of subject and marker, such that the image quality is still very good with regard to the representation. This is, for example, reasonable in the case of poor-contrast target subjects such as a specific region of patient tissue.

A 3D image data set of the patient can be generated as an image data set at a first point in time. The advance exposure of a 3D image data set includes the complete image information of a patient. Reconstructions of other viewing angles than were originally used for acquisition are also possible. It is possible at a second point in time to extract nearly any information from the image data set and to display it at the second point in time. During a treatment, information can thus also be shown that was not considered before the treatment or for which it was not expected that it would be required.

Primarily in the case of a 3D image data set, the registration per marker is important in order to determine both the correct perspective and the correct location of the information to be reconstructed from the 3D image data set given the later x-ray image acquired from an arbitrary (but registered, thus known and associable) direction.

A projection image in the viewing direction of the x-ray image can be reconstructed from the 3D image data set and can be displayed together with the x-ray image. A projection image that can be considered as supplementing the x-ray image is thus created that, aside from the current representation of subject and marker, provides all image information that would be made in an exposure of the patient at the second point in time according to the prior art. The same information is thus available to the doctor although the patient was irradiated with only a reduced dose at the second point in time.

A first partial image around the x-ray image and a second partial image can be extracted from the projection image, and both partial images can fused into a composite image and the composite image can be displayed.

The composite image as a product of the method thus comprises the merged information of the image data set and the x-ray image. The second partial image essentially extracts from the x-ray image the changes to which the subject to be represented is subject, with the subject being depicted in its currently applicable state. The remaining, time-variant information is extracted from the image data set in image form.

The fusion occurs such that both partial images are merged in order to yield a composite image as an x-ray image in which both partial images are merged in a spatially correct manner. An artificial x-ray image is thus created that corresponds to an x-ray image acquired with full dose according to the prior art at the second point in time.

The 3D image data set contains sufficient information in order, for example, to have available sufficient data material (given a yet unknown viewing direction of the x-ray image) in order to generate from the image data set a first partial image which appears in the viewing direction (previously unknown) of the x-ray image.

Every x-ray image can thus be supplemented with the supplementary data or surrounding data of the subject to be shown (which data of the patient is known in advance) from the 3D image data set in image form. The treating individual, for example in lithotripsy, is therewith given freedom at the second point in time to generate x-ray images of the subject to be shown in any viewing direction with a lower dose and to nevertheless supplement these with the time-invariant surrounding information to form full-fledged, artificially-generated x-ray images or composite images.

A calculus to be destroyed during a shockwave treatment can be represented as a subject. Given a calculus the requirements are directly given to be able to show the subject to be represented (namely the calculus) in sufficient image quality, even given a distinct dose reduction in the second x-ray image since said subject is clearly delimited from the surroundings in a radiological manner.

Moreover, in the case of a lithotripsy a complete 3D image data set of the patient is often produced (for example by computed tomography or by magnetic resonance tomography) some time before the beginning of the actual treatment. Primarily when the patient is located in a body position approximately corresponding to that for lithotripsy, the 3D image data which do not concern the subject to be represented retain their validity. To repeatedly map this information again via x-ray acquisitions is thus superfluous and is avoided by the inventive method. In the x-ray-intensive lithotripsy, only x-ray images with low dose are acquired at many different second points in time, namely during the treatment. The dose exposure of the patient thus accumulates to a very low value in comparison with conventional methods, but there is no information loss for the doctor does not hereby exist.

With regard to the apparatus, the object of the invention is achieved by an apparatus for x-ray imaging a patient containing a subject to be represented during a shockwave treatment, having a memory for an image data set generated at a first point in time and containing the subject and a marker; an x-ray system for acquisition of an x-ray image representing essentially only the subject and the marker at a second point in time, and with an evaluation unit for spatially-accurate association of the x-ray image with the image data set using the marker and for extraction of information from the image data set, and a display unit for display of the x-ray image together with the information during the shockwave treatment.

With the memory it is possible to provide the time-invariant information of the image data set for the later evaluation with regard to the information, or to store said time-invariant information up to the second point in time, or the acquisition of the x-ray image and the representation of the information. The x-ray system for the x-ray image can be made smaller, with less power and at a lower cost than the system for acquisition of the image data set since—as noted above—the x-ray image is to be acquired with a lower dose or smaller image field. A weaker-power x-ray system in turn produces advantages for the entire system since it is lighter, and thus the entire system (for example a C-arm supporting the x-ray system) can be produced with smaller dimensions, less complexity and more cost-effectively.

The evaluation unit can include a registration device for registration of image data set and x-ray image using the marker. The spatially-accurate association of x-ray image and image data set ensues with the registration device. The images thus do not have to first be adjusted to one another manually, for example, in the event that this is possible at all.

The image data set can be a 3D image data set. The apparatus then has an image processing system for reconstruction of a projection image from the 3D image data set.

The apparatus can include an image processing system for fusing a first partial image extracted from the projection image with a second partial image extracted from the x-ray image to form a composite image.

The image processing system can be, for example, a computer workstation with special computer software or can be a separately designed device.

The apparatus can be designed as an imaging subsystem of a shockwave system. As noted above, a shockwave system so equipped operates in a distinctly dose-reduced manner for the patient and medical personnel. As described above, the achieved treatment quality with the x-ray images to be acquired during the shockwave treatment is thereby obtained with unreduced image quality and/or image information.

The further advantages resulting from the apparatus have already been explained in connection with the inventive method.

### DESCRIPTION OF THE DRAWING

The single FIGURE schematically illustrates an embodiment of a workflow for x-ray imaging for a kidney stone lithotripsy in accordance with the invention.

### DESCRIPTION OF THE PREFERRED EMBODIMENTS

The FIGURE shows an exemplary workflow scenario for generation of an x-ray image 2 of a patient 4 during a kidney stone lithotripsy. The case history is that a patient 4 seeks out a doctor (not shown) and complains of abdominal pains. With an x-ray apparatus 6, the doctor (not shown) immediately produces an x-ray image 8 of the patient 4 by exposure of the patient with a standard dose of x-rays. In addition to the ribs 10 of the patient, his kidneys 12 with a kidney stone 14 located therein are visible on the x-ray image 8.

In order to confirm the initial diagnosis of the kidney stone 14 in the patient 4, the doctor arranges a further examination of the patient 4 at a later point in time 15. This corresponds to the inventive first point in time. By a computed tomography 16, a 3D image data set 18 containing a number of slice exposures 20 of the patient is hereby produced (likewise indicated by the arrow 5). The evaluation of the 3D image data set 18 confirms the initial diagnosis of the doctor, namely that the patient 4 suffers from a kidney stone 14.

The doctor hereupon arranges a shockwave lithotripsy which is to be subsequently conducted on the patient 4 in order to destroy the kidney stone 14. Indicated by the double arrow 21, the lithotripsy on the kidney stone 14 is conducted on the patient 4 by a lithotripter 22. The lithotripter 22 includes an x-ray image system 24 as a subsystem.

According to the prior art, a number of x-ray images (respectively with a standard dose of x-ray radiation) of the patient 4 would now be produced during the lithotripsy. All images would show both the kidney stone 14 in its respective current state of destruction and the kidney 12 and ribs 10 in an unchanged state. The patient would hereby be exposed to a high total x-ray dose.

By contrast, according to the invention a number of x-ray exposures of the patient 4 with a dose distinctly reduced relative to the standard dose are produced during the lithotripsy. The acquisition points in time 17 of these x-ray exposures respectively correspond to a second point in time of the inventive method. Of these produced x-ray images, one x-ray image 26 is shown as an example.

Since the x-ray image 26 would be acquired with distinctly reduced x-ray dose, for example relative to the x-ray image 8 or the 3D image data set, in the x-ray image 26 only the kidney stone 14 and parts of two ribs 10 of the patient 4 are directly visible in sufficient quality. The doctor conducting the kidney stone lithotripsy thus detects the present shape, size or degree of fragmentation of the kidney stone 14 in the x-ray image 26. To continue the kidney stone lithotripsy the doctor also requires information about the surrounding tissue (for example the kidney 12) or more information about the ribs 10 of the patient 4.

The doctor requires this information in order to know the positions of these anatomical items and to find suitable firing angles or locations for emitting the ultrasonic shockwaves into the patient 4. The doctor avoids hitting the ribs 10 or sensitive points of the kidney 12. This information, however, is not provided to the doctor by the x-ray image 26, or is not provided to a sufficient degree.

An image processing system 28 therefore extracts a partial x-ray image 20 which essentially comprises only the kidney stone 14. Since the x-ray image 26 was currently acquired, it also contains the current image information of the kidney stone 14.

Furthermore, from the 3D image data set 18 the image processing system 28 generates a projection x-ray image 32 with viewing direction and image segment corresponding to the x-ray image 26. The image processing system of a 3D image calculation system (not shown) that supplies the corresponding coordinate transformations between x-ray image system 24 and the 3D image data set works on this. For this, the navigation system resorts to characteristic points of the ribs 10 that thus serve as markers and associates x-ray image 26 and 3D image data set 18 with one another with spatial accuracy using the ribs 10.

In addition to the representation of the kidney stone 14, the projection x-ray image 32 also comprises the complete and high-quality representation of the ribs 10 and the kidney 12 of the patient 4 at the point in time April, thus the first point in time.

From the projection x-ray image 32, the image processing system 28 further generates a second partial x-ray image 34 that includes the entire image information of the projection x-ray image 32 with the exception of the kidney stone 14. Its previous representation obtained at the first point in time has in the meantime become out of date since it is destroyed and has already changed its position and shape. The remainder of the image information from the first point in time shown in the projection x-ray image is, however, also still valid at the time of lithotripsy. The reason for this is that the patient 4 adopted approximately the same support position for the exposure at the first point in time in the generation of the 3D image data set as now for the lithotripsy.

The image processing system 28 finally merges the two x-ray images 30 and 34 into the x-ray image 2 which now comprises the representation of the kidney 12 and the ribs 10 at the point in time of the generation of the 3D image data set in addition to the current representation of the kidney stone 14. It is thus a composite x-ray image. However, since neither shape nor position of the ribs 10 and the kidney 12 in the patient 4 have changed from the acquisition of the 3D image data set 18 to the implementation of the kidney stone lithotripsy (and therewith the acquisition of the x-ray image 26), the x-ray image 2 shows the artificial total representation of an x-ray image of the patient 4 which would actually have been acquired with high x-ray dose at the point in time of the x-ray image 26.

The complete exemplary embodiment in a current representation is thus available to the doctor although the patient 4 is only exposed with a significantly lower x-ray dose at the point in time of the x-ray acquisition 26. Since, as mentioned above, many further exposures are made during the lithotripsy in addition to the x-ray exposure 26, the x-ray dose is reduced multiple times relative to a method according to the prior art. Each of these exposures would have been conducted there with the standard dose.

To implement the aforementioned image processing steps, the image processing system 28 possesses an image storage 36 in which are stored or, respectively, buffered the corresponding images to be processed, for example the projection x-ray image 32 or the x-ray image 26. Here the image processing system 28 has access to the 3D image data set 18 via, for example, a network connection (not shown) to a hospital information system (likewise not shown). All image data of the appertaining patient 4 are archived there.

As an alternative to the procedure illustrated above, the partial x-ray image 34 can be directly generated from the x-ray image 8 acquired with the x-ray apparatus 6 at the earlier point in time. The requirement for this is merely that the patient 4 adopts approximately the same body position upon the acquisition of the x-ray image 8 as upon implementation of the kidney stone lithotripsy. Furthermore, it is a requirement that the x-ray exposure 8 was generated in the same acquisition direction or, respectively, viewing direction as the x-ray image 26. The selection of the image section corresponding to the x-ray image 26 and the positionally-accurate rotation of the x-ray image 8 are then effected by the image processing system 28.

As a further alternative, the partial x-ray image 34 can also be acquired with high x-ray dose via a one-time acquisition of an x-ray image 8 by the x-ray image system 23 in the lithotripter 22, indicated by the arrow 40. A current representation of the ribs 10 and the kidney 12 of the patient is hereby created in the x-ray image 8, namely temporally proximal to the generation of the x-ray image 26 and in the actual recumbent position of the patient 4 in the kidney stone lithotripsy. In this case, this first point in time and the second point in time cited below are separated by only a few minutes or hours, which is different than as above. The remaining proceeding x-ray exposures (corresponding to the x-ray image 26) during the kidney stone lithotripsy are then acquired again by the x-ray image system 24 with lower x-ray dose at the second points in time.

As an alternative or in addition to the acquisition of the x-ray image 26 with lower x-ray dose, the current acquisition of the kidney stone 14 can be implemented via selection of a smaller image section and therewith further dose reduction for the patient 4. A corresponding x-ray image 38 with current representation of the kidney stone 14 is shown dashed. The image section essentially overlaps only the surface of the kidney stone 14.

As an alternative to the merging of x-ray image 26 and image data set 18 described previously, only one item of supplementary information from the image data set 18 can also be displayed in the x-ray image 2. The x-ray image 2 then includes merely the image information of the x-ray image 26, but the current position coordinates of the kidney stone 14 in the apparatus are indicated in the apparatus coordinate system (not shown) of the lithotripter as information 42. Moreover, a position for the irradiation of the shockwave is specified that was calculated by the image processing system 28. The doctor thus does not need to evaluate the x-ray image 2 himself in order to determine the correct radiation direction or, respectively, the radiation location for the launching of the shockwave head.

All images and information cited above can be displayed to the doctor or the like separately or together on image monitors (for example computer monitors 44 of the image processing system 28) as a display unit.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for imaging a patient containing a subject to be represented during a shockwave treatment, comprising the steps of:
at a first point in time, generating an x-ray image data set representing a portion of a patient and a subject contained within the patient and a marker;
at a second point in time subsequent to said first point in time, generating an x-ray image representing substantially only the subject and the marker;
correctly spatially associating said x-ray image with said x-ray image data set using said marker; and
during a shockwave treatment, displaying said x-ray image together with information extracted from said image data set.

2. A method as claimed in claim 1 comprising displaying coordinates identifying a spatial position of said subject within said patient as said information.

3. A method as claimed in claim 1 comprising displaying a limited environment of said subject within said patient as said information.

4. A method as claimed in claim 1 comprising generating said x-ray image with a lower radiation dose than was used for generating said image data set, said lower radiation dose still allowing said subject and said marker to be immediately visible in said x-ray image.

5. A method as claimed in claim 1 comprising generating said x-ray image with a smaller image field, that contains said subject and said marker, than an image field for said image data set.

6. A method as claimed in claim 1 comprising generating said image data set as a 3D image data set.

7. A method as claimed in claim 6 comprising reconstructing a projection image from said 3D image data set, and displaying said projection image together with said x-ray image.

8. A method as claimed in claim 7 comprising extracting a first partial image from said projection image and fusing said first partial image with a second partial image extracted from said x-ray image to form a composite image, and displaying said composite image.

9. A method as claimed in claim 1 comprising generating said image data set and said x-ray image to show a calculus in the patient as said subject.

10. An apparatus for imaging a patient containing a subject to be represented during a shockwave treatment, comprising:
a memory containing a stored x-ray image data set generated at a first point in time, representing a portion of a patient and a subject contained within the patient and a marker;
an x-ray imaging system that, at a second point in time subsequent to said first point in time, generates an x-ray image representing substantially only the subject and the marker;
a computer that correctly spatially associates said x-ray image with said x-ray image data set using said marker; and
during a shockwave treatment, displaying said x-ray image together with information extracted from said image data set.

11. An apparatus as claimed in claim 10 wherein said display unit displays coordinates identifying a spatial position of said subject within said patient as said information.

12. An apparatus as claimed in claim 10 wherein said display unit displays a limited environment of said subject within said patient as said information.

13. An apparatus as claimed in claim 10 wherein said x-ray imaging system generates said x-ray image with a lower radiation dose than was used for generating said image data set, said lower radiation dose still allowing said subject and said marker to be immediately visible in said x-ray image.

14. An apparatus as claimed in claim 10 wherein said x-ray imaging system generates said x-ray image with a smaller image field, that contains said subject and said marker, than an image field for said image data set.

15. An apparatus as claimed in claim 10 wherein said image data set is a 3D image data set, and comprising an image processing system that reconstructs a projection image from said 3D image data set, and wherein said display unit displays said projection image together with said x-ray image.

16. An apparatus as claimed in claim 15 comprising wherein said image processing system extracts a first partial image from said projection image and fuses said first partial image with a second partial image extracted from said x-ray image to form a composite image, and wherein said display unit displays said composite image.
